(19) United States
(12) Patent Application Publication
(10) Pub. No.: US 2024/0290450 A1
(43) Pub. Date: Aug. 29, 2024

(54) INFORMATION PROCESSING SYSTEM, INFORMATION PROCESSING METHOD, AND COMPUTER READABLE MEDIUM

(71) Applicant: NEC Corporation, Tokyo (JP)

(72) Inventor: Atsushi SHIBATA, Tokyo (JP)

(73) Assignee: NEC Corporation, Tokyo (JP)

(21) Appl. No.: 18/581,489

(22) Filed: Feb. 20, 2024

(30) Foreign Application Priority Data
Feb. 28, 2023 (JP) 2023-029542

(51) Int. Cl. G16H 20/10; G06V 40/20; G16H 80/00

(52) U.S. Cl. CPC G16H 20/10; G06V 40/28; G16H 80/00

(57) ABSTRACT

In an information processing system, a first information acquisition unit acquires first information including a name of a medicine. A second information adding unit adds second information about the medicine associated with the first information to the first information. A presentation unit presents, to a user, the first and second information in association with each other. A conversion unit shows, upon receiving an operation indicating the second information performed by the user, the first information associated with the second information to a communication destination.

### INCORPORATION BY REFERENCE

This application is based upon and claims the benefit of priority from Japanese patent application No. 2023-029542, filed on Feb. 28, 2023, the disclosure of which is incorporated herein in its entirety by reference.

### TECHNICAL FIELD

The present disclosure relates to an information processing system, an information processing method, and a computer readable medium.

### BACKGROUND ART

A person who has difficulty in hearing may have difficulty in smoothly communicating on certain topics in which technical terms are used. For example, when a user, i.e., a consumer, purchases a medicine at a pharmacy, he or she may consult with a pharmacist or the like about the medicine or about health conditions related to the medicine. There have been proposals to facilitate communication in such situations.

For example, a system disclosed in Published Japanese Translation of PCT International Publication for Patent Application, No. 2019-535059 recognizes and interprets sign language, and presents translated information to a user of an MR (Mixed Reality) device.

Further, in a system disclosed in International Patent Publication No. WO2021/006094, a user carries out a search by saying appearance information in addition to (or instead of) a code and/or name of a medicine, and thereby performs a search based on the appearance information even when the code and name are unknown.

### SUMMARY

However, there is a demand for a technology that enables a person having difficulty in hearing to communicate with other persons or the like more smoothly.

In view of the above-described problem, an object of the present disclosure is to provide an information processing system and the like for preventing the stagnation of communication.

An information processing system according to the present disclosure includes a first information acquisition unit, a second information adding unit, a presentation unit, and a conversion unit. The first information acquisition unit acquires first information including a name of a medicine. The second information adding unit adds second information about the medicine associated with the first information to the first information. The presentation unit presents, to a user, the first and second information in association with each other. The conversion unit shows, upon receiving an operation indicating the second information performed by the user, the first information associated with the second information to a communication destination.

In an information processing method according to the present disclosure, a computer performs the following method. The computer acquires first information including a name of a medicine that a user intends to obtain. The computer adds second information about the medicine associated with the first information to the first information. The computer displays the first and second information in association with each other. The computer shows, upon receiving an operation indicating the second information performed by the user, the first information associated with the second information to a communication destination.

A program according to the present disclosure causes a computer to perform the following method. The computer acquires first information including a name of a medicine that a user intends to obtain. The computer adds second information about the medicine associated with the first information to the first information. The computer displays the first and second information in association with each other. The computer shows, upon receiving an operation indicating the second information performed by the user, the first information associated with the second information to a communication destination.

### BRIEF DESCRIPTION OF DRAWINGS

The above and other aspects, features and advantages of the present disclosure will become more apparent from the following description of certain example embodiments when taken in conjunction with the accompanying drawings, in which:

FIG. 1 is a block diagram of an information processing system according to a first example embodiment;

FIG. 2 is a flowchart of an information processing method according to the first example embodiment;

FIG. 3 shows an example of use of an information processing system according to a second example embodiment;

FIG. 4 is a block diagram of an information processing system according to the second example embodiment;

FIG. 5 shows an example of a prescription;

FIG. 6 shows an example of information displayed on a display;

FIG. 7 shows an example of processes performed by a conversion unit;

FIG. 8 shows an example of use of an information processing system according to a third example embodiment;

FIG. 9 is a block diagram of an information processing system according to the third example embodiment; and

FIG. 10 is a block diagram showing an example of a hardware configuration of a computer.

### EXAMPLE EMBODIMENT

The present disclosure will be described hereinafter through an example embodiment of the disclosure, but the disclosure according to the claims is not limited to the below-shown example embodiment. Further, all the components/structures described in the example embodiment are not necessarily indispensable as means for solving the problem. Note that for clarifying the explanation, the following description and the drawings are partially omitted and simplified as appropriate. Note that the same reference numerals (or symbols) are assigned to the same elements throughout the drawings, and redundant descriptions thereof are omitted as appropriate.

### First Embodiment

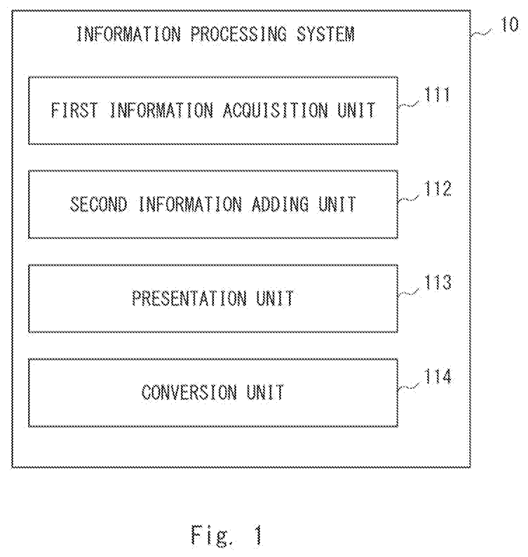

A first example embodiment will be described hereinafter. FIG. 1 is a block diagram of an information processing system 10 according to the first example embodiment. The information processing system 10 is a system for enabling a pharmacist or the like and a user to communicate with each other about a medicine.

The pharmacist or the like in the present disclosure refers to a pharmacist who prepare a medicine or a person who provides a medicine prepared by a pharmacist to a user. The pharmacist or the like may include a doctor who prescribes a medicine. Note that in the following description in the present disclosure, the pharmacist or the like is also simply referred to as a pharmacist. The user in the present disclosure is a consumer who receives a medicine prepared by a pharmacist. The user may be a person who takes the medicine or a person who communicates with a pharmacist on behalf of a person who takes the medicine.

The information processing system 10 is installed, for example, at the counter of a pharmacy, and assists a pharmacist and a user in having their conversation. The information processing system 10 includes, as its main configuration, a first information acquisition unit 111, a second information adding unit 112, a presentation unit 113, and a conversion unit 114.

The first information acquisition unit 111 acquires first information. The first information includes at least the name of a medicine that the user takes. The first information may include other information about the medicine in addition to the name of the medicine. The other information about the medicine may include, for example, the usage (e.g., how to take the medicine), the dosage, and the like when the user takes the medicine. When the medicine has an identification code, the first information may include the identification code. When the information processing system 10 receives a plurality of names of medicines, it acquires each of these names as a separate piece of first information different from the other pieces of first information.

For example, the first information acquisition unit 111 may receive an operation performed by a pharmacist and acquire first information from information entered by the pharmacist. For example, the first information acquisition unit 111 may receive information about a prescription that a user has received from a doctor, and acquire first information from the received information about the prescription.

The second information adding unit 112 adds second information about the medicine associated with the first information to the first information. The second information is information individually added to the name of the medicine included in the first information, or individually added to each piece of the other information about the medicine included in the first information. The second information is information that is added to the first information to identify the first information in communication between the pharmacist and the user.

In some cases, the name of a medicine included in the first information is not familiar to the user. Further, the name of a medicine included in the first information may be difficult for a person, whose hearing is impaired, to express by using sign language. It is preferable that the second information is information that can be easily used as a substitute for the first information in communication. The second information may be, for example, a code associated with the first information. The second information may be, for example, an abbreviation of the first information. Alternatively, the second information may be a substitutive alias of the first information.

The presentation unit 113 presents, to the user, the first and second information in association with each other. The presentation unit 113 displays the first and second information, for example, on a display connected to the information processing system 10.

When the conversion unit 114 receives an operation indicating the second information performed by the user, it shows the first information associated with the second information to the communication destination. That is, when the user intends to communicate with the pharmacist or the like by using the second information, the conversion unit 114 shows the first information associated with the second information to the person with whom the user intends to talk. When doing so, the conversion unit 114 receives an operation or action performed by the user and recognizes the second information from the received operation or action. The conversion unit 114 converts the recognized second information into the first information and shows the obtained first information to the communication destination (or with which the user intends to talk). In this case, the communication destination (or with which the user intends to talk) is the pharmacist.

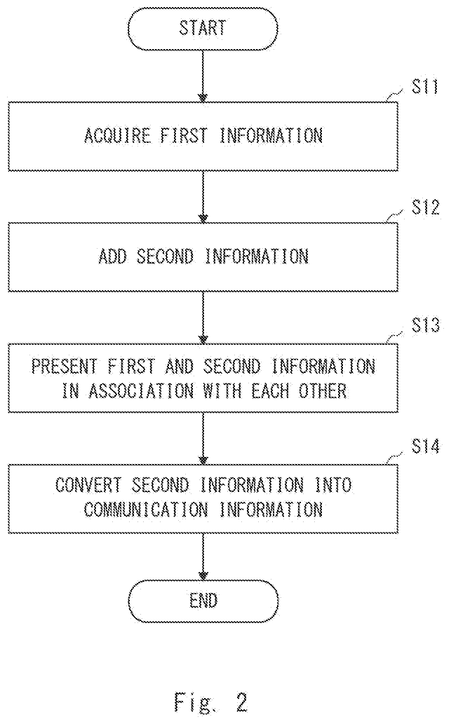

Next, processes performed by the information processing system 10 will be described. FIG. 2 is a flowchart of an information processing method according to the first example embodiment. A series of processes shown in FIG. 2 is started when, for example, the information processing system 10 receives first information.

Firstly, the first information acquisition unit 111 acquires first information including the name of a medicine that a user intends to obtain (Step S11). The first information acquisition unit 111 supplies the acquired first information to the second information adding unit 112.

Next, the second information adding unit 112 adds second information about the medicine associated with the first information to the first information (Step S12). The second information adding unit 112 supplies the first and second information to the presentation unit 113.

Next, the presentation unit 113 displays the first and second information in association with each other (Step S13).

Next, when conversion unit 114 receives an operation indicating second information performed by the user, it shows the first information associated with the second information to the communication destination (Step S14).

The information processing method performed by the information processing system 10 has been described above. Through the above-described processes, the information processing system 10 can perform the information processing method for preventing communication from being stopped or becoming unable to be continued.

Note that the information processing system 10 may have a processor and a storage device as a configuration which is not shown. Examples of the storage device included in the information processing system 10 include storage devices including, for example, non-volatile memories such as a flash memory or an SSD (Solid State Drive). In this case, in the storage device included in the information processing system 10, a computer program (hereinafter also referred to simply as a program) for performing the above-described image processing method is stored. Further, the processor loads the computer program from the storage device onto a buffer memory such as a DRAM (Dynamic Random Access Memory) and performs the loaded program.

Each component of the information processing system 10 may be implemented by a dedicated hardware. Further, some or all of the components may be implemented by general-purpose or dedicated circuitry, a processor, or a combination thereof. These components may be formed by a single computer chip or by a plurality of computer chips connected to each other through a bus. Some or all of the components of each apparatus may be implemented by a combination of the above-mentioned circuitry or the like and a program(s). Further, a CPU (Central Processing Unit), a GPU (Graphics Processing Unit), an FPGA (Field-Programmable Gate Array), or the like may be used as the processor. Note that the description about the configuration described here may also be applied to other apparatuses or systems described below in the present disclosure.

Further, when some or all of the components of the information processing system 10 are implemented by a plurality of information processing apparatuses, circuits, or the like, the plurality of information processing apparatuses, circuits, or the like may be disposed at one place in a concentrated manner or distributed over a plurality of places. For example, the information processing apparatuses, circuits, or the like may be implemented in the form of a client-server system, a cloud computing system, or the like in which components or the like are connected to each other through a communication network. Further, the functions of the information processing system 10 may be provided in the form of SaaS (Software as a Service).

According to the first example embodiment as described above, it is possible to provide an information processing system, an information processing method, and a program for preventing communication from being stopped or becoming unable to be continued.

### Second Example Embodiment

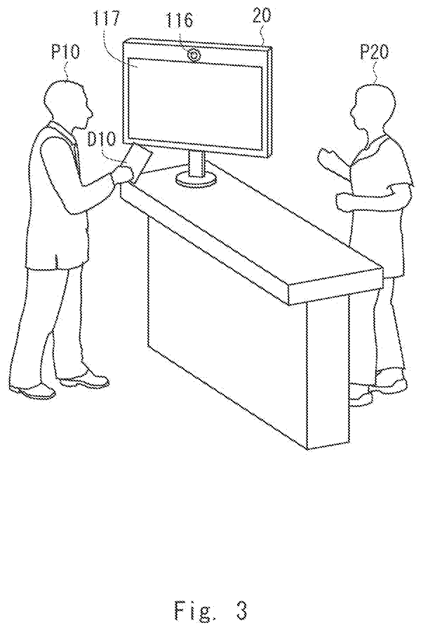

Next, a second example embodiment will be described. FIG. 3 shows an example of use of an information processing system according to the second example embodiment. FIG. 3 shows a situation in which, in s pharmacy, a user P10, who have visited the pharmacy, and a pharmacist P20 communicate with each other through an information processing system 20. The user P10 brings a prescription D10 prescribed by a doctor to the pharmacy and intends to obtain a prescribed medicine. The pharmacist P20 explains the medicine to be provided. Alternatively, the user P10 asks questions about the medicine to be provided. When doing so, the user P10 and the pharmacist P20 communicate with each other while looking at information displayed by the information processing system 20.

The information processing system 20 includes a camera 116 and a display 117. The camera 116 may photograph the prescription brought by the user P10 and thereby generate image data thereof. Further, when the user P10 communicates with the pharmacist or the like through sign language, the camera 116 may take an image of sign language. In this case, the information processing system 20 converts the image of the sign language into certain communication information. The certain communication information is text information, voice information, or the like that the pharmacist, whom the user is talking with, can recognize. The pharmacist P20 can understand the intention of the user P10 by recognizing the communication information. Further, the pharmacist P20 displays information about the medicine on the information processing system 20, and explains the medicine while presenting the displayed information to the user P10.

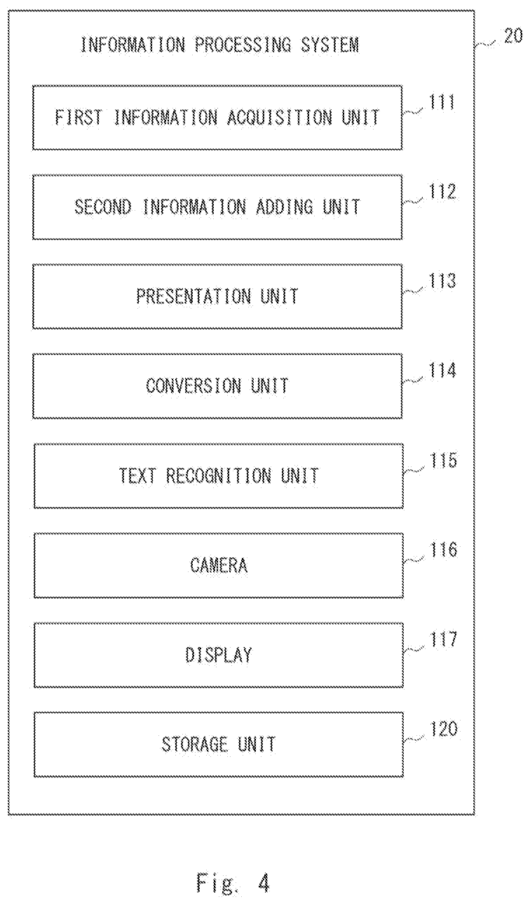

Next, the information processing system 20 will be described with reference to FIG. 4. FIG. 4 is a block diagram of the information processing system 20 according to the second example embodiment. The information processing system 20 includes, as its main configuration, a first information acquisition unit 111, a second information adding unit 112, a presentation unit 113, a conversion unit 114, a text recognition unit 115, a camera 116, a display 117, and a storage unit 120.

The first information acquisition unit 111 according to this example embodiment acquires first information from the information about the prescription D10 presented by the user P10. More specifically, when the camera 116 of the information processing system 20 photographs the prescription D10, the first information acquisition unit 111 acquires the first information from the image data thereof. In this process, the text recognition unit 115 recognizes text from the prescription D10.

The first information acquisition unit 111 may acquire first information from a prescription prescribed to the user, a medication history of the user, or product information of a medicine that the user wants to purchase. That is, for example, the first information acquisition unit 111 may acquire first information from a product package or an advertisement for a medicine instead of acquiring it from the prescription. Further, the first information acquisition unit 111 may acquire, as the first information, at least one of the name of a medicine, the dosage of the medicine, and the usage of the medicine.

The second information adding unit 112 according to this example embodiment adds second information to the first information. Note that the second information adding unit 112 reads second information from a second information database stored in the storage unit 120 and adds the read second information to the first information. The second information adding unit 112 may add, as the second information, a unique identifier to the first information.

The presentation unit 113 displays the first and second information on the display 117 in a state in which the first and second information are associated with each other. In addition to the first and second information, the presentation unit 113 may display the usage of the medicine, the dosage of the medicine, and other precautions on the display 117.

The conversion unit 114 converts an image of a gesture made (or performed) by the user P10 into communication information associated with the gesture. In this case, the gesture may mean (i.e., indicate), for example, an operation to select the second information to be displayed on the display 117. Further, the conversion unit 114 may convert an image of sign language image indicated (or performed) by the user into communication information. That is, when the camera 116 photographs a gesture of the user, the conversion unit 114 recognizes second information from the photographed image of the gesture, converts the recognized second information into the first information, and shows the obtained first information to the communication destination. In this example embodiment, the conversion unit 114 displays the first information on the display 117 in such a manner that the pharmacist P20 can recognize the first information.

The conversion unit 114 may convert an image of sign language indicated (or performed) by the user P10 into text information or voice information as the communication information. In this case, the purpose of the text information or voice information obtained by the conversion unit 114 is to enable the pharmacist P20 to recognize the image of the sign language.

Note that as the technology for reading sign language from an image of the sign language and converting the read sign language into text data, a technology well-known to those skilled in the art can be adopted. Therefore, its details are omitted in the present disclosure.

As an example, the conversion unit 114 may convert an image of the user into text data by analyzing the image using a machine learning technique. More specifically, for example, the camera 116 acquires an image of an operation performed by the user. The conversion unit 114 infers sign language corresponding to second information from the image of the operation performed by the user, acquired by the camera, and converts the second information into the first information based on the image of the inferred sign language. In this way, the information processing system 20 can enable the user and the pharmacist to communicate with each other by using a communication method optimized for the user.

The text recognition unit 115 recognizes text from data of an image taken by the camera 116. Specifically, the text recognition unit 115 recognizes, for example, text written or printed in the prescription from image data obtained by photographing the prescription presented by the user P10.

The camera 116 is a photographing apparatus that is configured so as to be able to photograph the prescription presented by the user P10, a gesture performed by the user P10, or the like. The display 117 is, for example, a liquid-crystal display panel or a display device including organic electroluminescence, and enables the user P10 and the pharmacist P20 to recognize various kinds of information in the form of images.

The storage unit 120 is a storage device examples of which include a non-volatile memory such as a flash memory and an SSD (Solid State Drive). In the storage unit 120, certain communication information associated with the first information is stored in advance. The certain communication information is, for example, information about a certain sign language gesture that can be added to the first information. In this case, the second information adding unit 112 selects certain communication information associated with the first information and adds the selected communication information to the first information. In this way, for example, the information processing system 20 displays, for the user P10, a sign language gesture corresponding to the first information, which is the name of the medicine, as the second information.

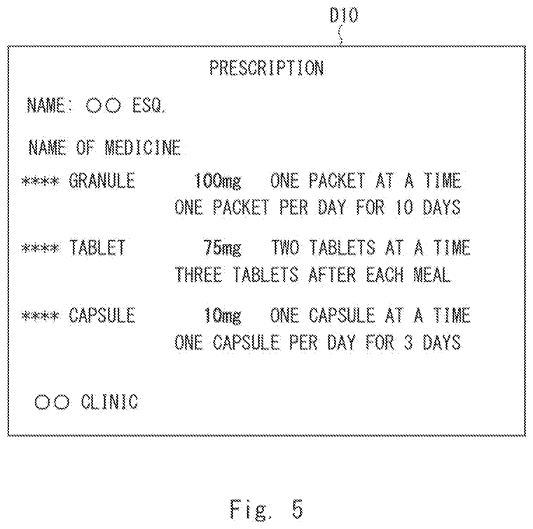

Next, information written or printed in the prescription will be described with reference to FIG. 5. FIG. 5 shows an example of a prescription. FIG. 5 shows a prescription D10. The prescription D10 includes the name of the user P10 who obtains the medicine and the name of the prescribed medicine (the name of the medicine). Further, the prescription D10 includes the dosage, usage, and the like of the medicine, that accompany the name of the medicine.

For example, when the user P10 receives such a prescription D10 from a doctor, he/she brings the received prescription D10 to a pharmacy and presents it to a pharmacist. When doing so, for example, the user P10 can make the information processing system 20 read the prescription D10. Note that the user P10 may pass the prescription D10 directly to the pharmacist P20. In this case, the pharmacist P20 may enter information about the medicine to be provided to the user P10 into the information processing system 20.

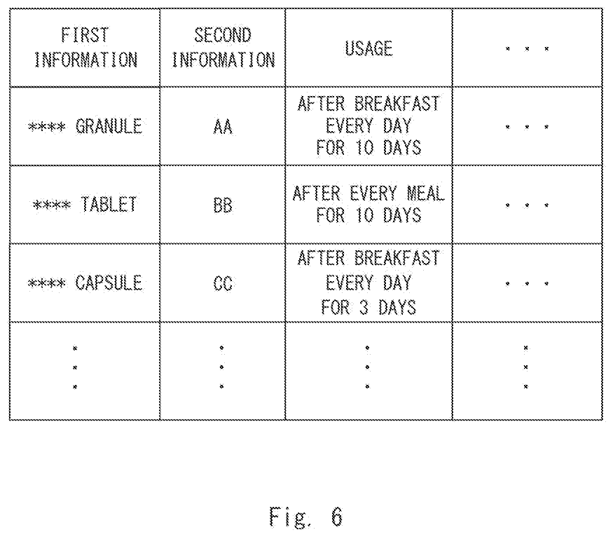

Next, an example in which first and second information are displayed together will be described with reference to FIG. 6. FIG. 6 shows an example of information displayed on a display. The table shown in FIG. 6 is generated so that the pharmacist P20 can present information about the medicine to the user P10. In the table shown in FIG. 6, in each row, the name of a medicine is shown as first information, and second information associated with the first information is shown next to the first information. Further, the usage and the like of a respective medicine are shown in each row in the table shown in FIG. 6. The user P10 and the pharmacist P20 communicate with each other while looking at this information.

Specifically, in FIG. 6, for example, “**** granule” is indicated as first information; “AA” is indicated as second information corresponding thereto; and “after breakfast every day” and “for 10 days” are indicated as usage. Similarly, in FIG. 6, in the row below the row of the above-described information, “**** tablet” is indicated as first information; “BB” is indicated as second information; and “after every meal” and “for 10 days” are indicated as usage. Further, in FIG. 6, “**** capsule” is indicated as first information; “CC” as indicated as second information; and “after breakfast every day” and “for 3 days” are indicated as usage.

The user P10 can visually recognize these pieces of information and perform sign language by using “AA” instead of using “**** granule”. Similarly, the user P10 can perform sign language by using “BB” instead of using “**** tablet”, and can perform sign language by using “CC” instead of using “**** capsule”.

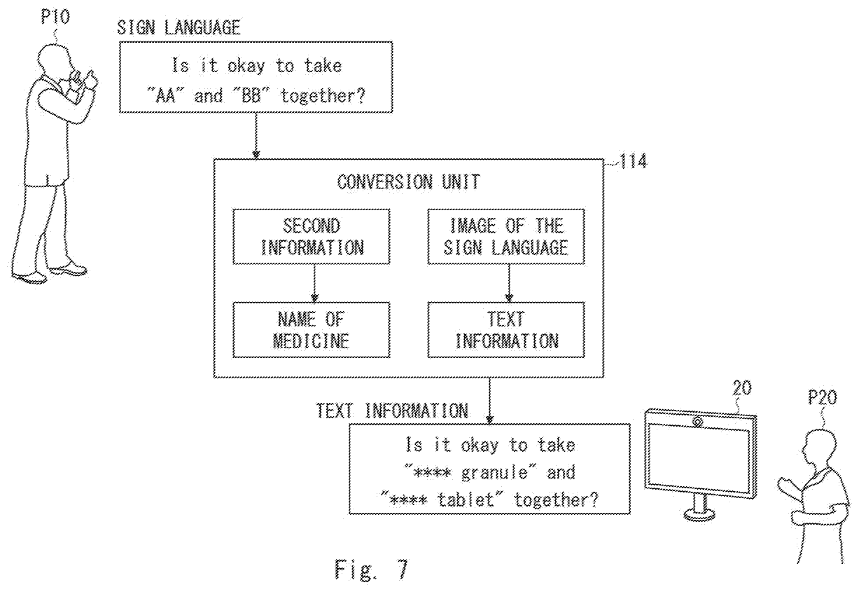

The function of the conversion unit 114 will be described with reference to FIG. 7. FIG. 7 shows an example of processes performed by the conversion unit. FIG. 7 shows information that the conversion unit 114 receives, processes that the conversion unit 114 performs, and information that the conversion unit 114 outputs.

Firstly, the conversion unit 114 receives a sign language gesture performed by the user P10 in the form of image data. For example, the user P10 expresses “Is it okay to take AA and BB together?” by a gesture using second information. Upon receiving image data of this gesture, the conversion unit 114 converts the image of the sign language into text information. When second information is included in the obtained text information, the conversion unit 114 converts this second information into the first information. Then, the conversion unit 114 generates a message indicating that “Is it okay to take the **** granule and the **** tablet together?” as the text information obtained by converting the second information into the first information, and then displays the generated message on the display 117.

In this way, the user P10 can smoothly express his/her intention by using the second information without using sign language expressing a technical expression, i.e., expressing the actual name of the medicine. Therefore, the user P10 can smoothly consult with the pharmacist or the like about the medication or the like. Further, the pharmacist P20 can recognize the message of the user P10 as well as the actual name of the medicine based on the first information. Further, the pharmacist P20 can communicate with the user P10 through the information processing system 20 without any trouble or delay even when he/she cannot understand the sign language. That is, by the above-described configuration, the information processing system 20 can prevent communication from being stopped or becoming unable to be continued.

While the second example embodiment has been described above, the information processing system 20 is not limited to the above-described configuration. The information processing system 20 may include, for example, a scanner for reading a prescription D10. The information processing system 20 may include a display by which the user P10 visually recognize information, and a display by which the pharmacist P20 visually recognize information. The information processing system 20 may also include an information input device such as a touch panel or a keyboard. Further, all the components of the information processing system 20 may be formed as one integrated apparatus or the like, or may be configured in a state in which a plurality of apparatuses are connected to each other so that they communicate with each other.

As described above, according to this example embodiment, it is possible to provide an information processing system, an information processing method, and a program for preventing communication from being stopped or becoming unable to be continued.

### Third Example Embodiment

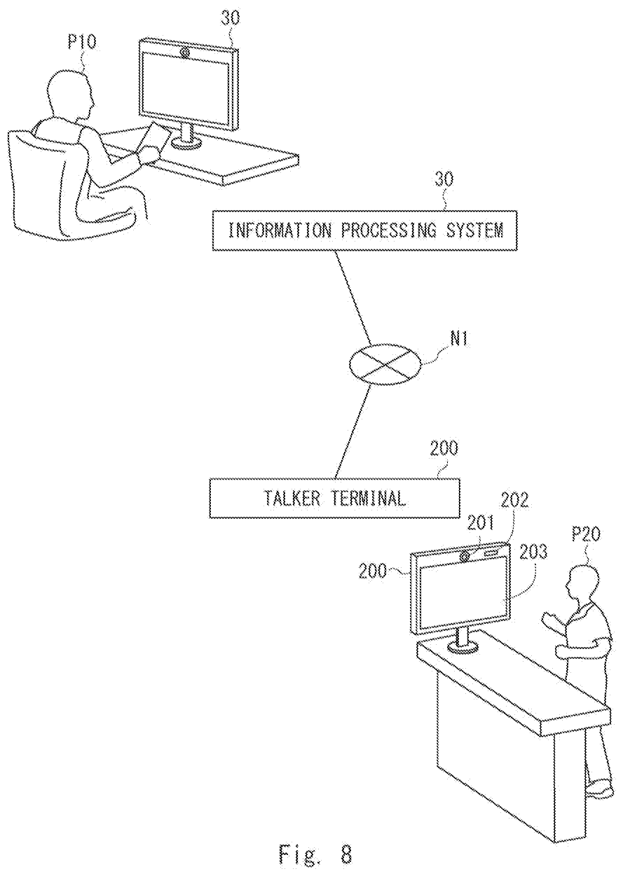

Next, a third example embodiment will be described. FIG. 8 shows an example of use of an information processing system 30 according to the third example embodiment. The information processing system 30 differs from the information processing system according to the second example embodiment in that the information processing system 30 is configured so that a user P10 and a pharmacist P20 can communicate with each other from their respective places remote from each other.

In FIG. 8, the user P10 shown in an upper part thereof uses the information processing system 30, for example, in his/her house. The information processing system 30 is connected to a network N1 so that it can communicate with other systems or the like. Further, in a pharmacy shown in a lower part of FIG. 8, the pharmacist P20 uses a talker terminal 200 that is connected to the information processing system 30 through the network N1 so that they can communicate with each other.

Specifically, examples of the talker terminal 200 include a notebook PC (Personal Computer) equipped with a communication function, a desktop PC equipped with the same, a tablet-type terminal equipped with the same, and a smartphone equipped with the same. The talker terminal 200 includes a camera 201, a microphone 202, and a display 203, all of which are controlled by a control apparatus such as a CPU.

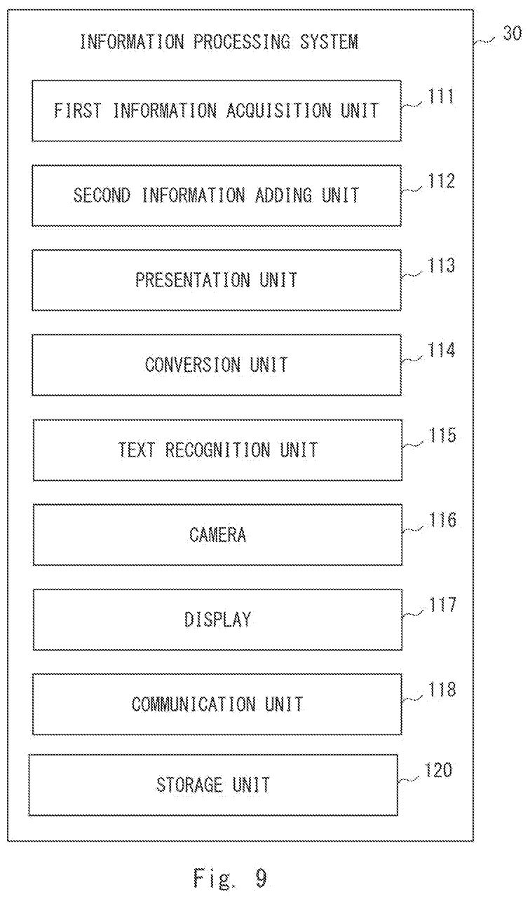

The information processing system 30 will be described with reference to FIG. 9. FIG. 9 is a block diagram of the information processing system 30 according to the third example embodiment. The information processing system 30 differs from the information processing system 20 in that the information processing system 30 includes a communication unit 118.

The communication unit 118 is an interface unit for enabling the information processing system 30 to communicate with the talker terminal 200. For example, the communication unit 118 may transmit an image of a prescription photographed by the camera 116 to the talker terminal 200. When the text recognition unit 115 generates text information from the image of the prescription, the communication unit 118 may transmit the generated text information to the talker terminal 200. Further, the communication unit 118 receives information about a medicine transmitted by the pharmacist P20 through the talker terminal 200, and supplies the received information to each component of the information processing system 30.

By the above-described configuration, the user P10 can communicate with the pharmacist P20 about the medicine at a place remote from the place of the pharmacist P20. When doing so, the information processing system 30 adds second information to the first information. Therefore, the user P10 can communicate with the pharmacist P20 by using the second information.

The third example embodiment has been described above. According to this example embodiment, it is possible to provide an information processing system, an information processing method, and a program for preventing communication from being stopped or becoming unable to be continued. Note that the present disclosure is not limited to the above-described guidance about medication, but can also be applied to cases where communication is performed for other types of articles. That is, in the present disclosure, for example, the first information acquisition unit may acquire first information including the name of a product, and the second information adding unit add second information about the product associated with the first information to the first information. Further, in this case, the presentation unit may present, to the user, the first and second information in association with each other. Further, when the conversion unit receives an operation indicating second information performed by the user, the presentation unit may show the first information associated with the second information to the communication destination. Further, the conversion unit may convert an image of a gesture made (e.g., performed) by the user into communication information associated with the gesture, and/or may convert an image of sign language indicated (e.g., performed) by the user into communication information. By the above-described configuration, the information processing system can prevent communication for explaining a product from being stopped or becoming unable to be continued.

Note that the aforementioned program may be stored in various types of non-transitory computer readable media and thereby supplied to computers. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Read Only Memory), CD-R, CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

### Example of Hardware Configuration

A case in which each functional component of an information processing system according to the present disclosure is implemented by a combination of hardware and software will be described hereinafter.

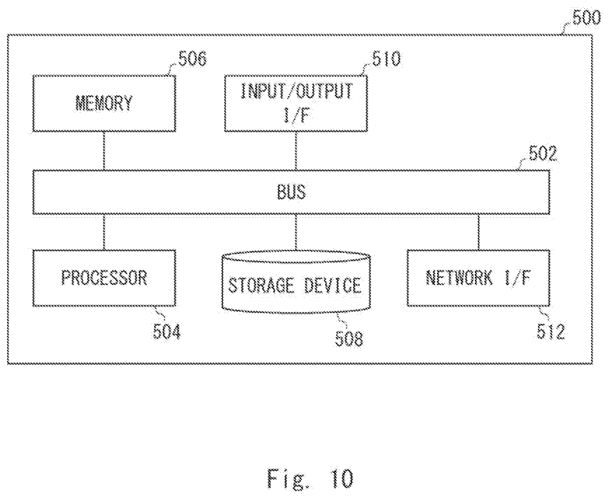

FIG. 10 is a block diagram showing an example of a hardware configuration of a computer. The information processing system according to the present disclosure can implement the above-described functions by a computer 500 including the hardware configuration shown in FIG. 10. The computer 500 may be a portable computer such as a smartphone or a tablet-type terminal, or may be a stationary computer such as a PC. The computer 500 may be a dedicated computer designed to implement a respective apparatus, or may be a general-purpose computer. The computer 500 can implement desired functions by installing therein a predetermined application(s).

The computer 500 include a bus 502, a processor 504, a memory 506, a storage device 508, an input/output interface (I/F) 510 and a network interface (I/F) 512. The bus 502 is a data transmission path through which the processor 504, the memory 506, the storage device 508, the input/output interface 510, and the network interface 512 transmit and receive data to and from each other. However, the method for connecting the processor 504 and the like to each other is not limited to connections through buses.

The processor 504 is any of various types of processors such as a CPU, a GPU, or an FPGA. The memory 506 is a main storage device implemented by using a RAM (Random Access Memory) or the like.

The storage device 508 is an auxiliary storage device implemented by using a hard disk, an SSD, a memory card, a ROM (Read Only Memory), or the like. In the storage device 508, a program(s) for implementing desired functions is stored. The processor 504 implements each of functional components of each apparatus by loading this program onto the memory 506 and executing the loaded program.

The input/output interface 510 is an interface for connecting the computer 500 with an input/output device(s). For example, an input device such as a keyboard and an output device such as a display device are connected to the input/output interface 510.

The network interface 512 is an interface for connecting the computer 500 to a network.

Although the present disclosure is described above with reference to example embodiments, the present disclosure is not limited to the above-described example embodiments. Various modifications that can be understood by those skilled in the art can be made to the configuration and details of the present disclosure within the scope of the disclosure.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

According to the present disclosure, it is possible to provide an information processing system, an information processing method, and a program for preventing communication from being stopped or becoming unable to be continued.

What is claimed is:

1. An information processing system comprising:
A first information acquisition unit configured to acquire first information including a name of a medicine;
a second information adding unit configured to add second information about the medicine associated with the first information to the first information;
a presentation unit configured to present, to a user, the first and second information in association with each other; and
a conversion unit configured to show, upon receiving an operation indicating the second information performed by the user, the first information associated with the second information to a communication destination.

2. The information processing system according to claim 1, wherein the conversion unit converts an image of a gesture made by the user into communication information associated with the gesture.

3. The information processing system according to claim 2, wherein the conversion unit converts an image of sign language indicated by the user into the communication information.

4. The information processing system according to claim 3, wherein the conversion unit converts, as the communication information, the image of the sign language indicated by the user into text information or voice information.

5. The information processing system according to claim 1, wherein the first information acquisition unit acquires the first information from a prescription prescribed to the user, a medication history of the user, or product information of a medicine that the user wants to purchase.

6. The information processing system according to claim 1, wherein the first information acquisition unit acquires, as the first information, at least one of a name of a medicine, a dosage of the medicine, and usage of the medicine.

7. The information processing system according to claim 1, wherein the second information adding unit adds a unique identifier to the first information as the second information.

8. The information processing system according to claim 1, further comprising a storage unit configured to store certain communication information associated with the first information in advance, wherein
the second information adding unit selects the communication information and adds the selected communication information to the first information.

9. An information processing method wherein a computer:
acquires first information including a name of a medicine that a user intends to obtain;
adds second information about the medicine associated with the first information to the first information;
displays the first and second information in association with each other; and
shows, upon receiving an operation indicating the second information performed by the user, the first information associated with the second information to a communication destination.

10. A non-transitory computer readable medium storing a program for causing a computer to perform an information processing method comprising:
acquiring first information including a name of a medicine that a user intends to obtain;
adding second information about the medicine associated with the first information to the first information;
displaying the first and second information in association with each other; and
showing, upon receiving an operation indicating the second information performed by the user, the first information associated with the second information to a communication destination.

11. The information processing system according to claim 1, wherein the conversion unit infers the second information from an image of an operation performed by the user by using a machine learning technique, and shows the first information associated with the second information to the communication destination so as to enable the user and the pharmacist to communicate with each other by using a communication method optimized for the user.
